(19) United States
(12) Patent Application Publication
(10) Pub. No.: US 2019/0053941 A1
(43) Pub. Date: Feb. 21, 2019

(54) SYSTEM FOR REDUCING LOCALISED FATTY MASSES BY MEANS OF COLD APPLICATION, APPLICATOR FOR SUCH A SYSTEM AND NON-INVASIVE TREATMENT METHOD FOR REDUCING FATS BY MEANS OF COLD APPLICATION

(71) Applicant: DELEO, Saint Raphaël (FR)

(72) Inventor: Herbert SAMSON, Saint Raphael (FR)

(21) Appl. No.: 16/089,393

(22) PCT Filed: Mar. 31, 2017
(86) PCT No.: PCT/FR2017/050747

§ 371 Date: Sep. 28, 2018

(30) Foreign Application Priority Data
Mar. 31, 2016 (FR) 16 52798

(51) Int. Cl. A61F 7/02; A61F 7/10

(52) U.S. Cl. CPC A61F 7/02; A61F 2007/0056; A61F 7/10

(57) ABSTRACT

A non-invasive treatment method for reducing fats using a system (10) for performing a non-invasive treatment for reducing fats by cold application. The system (10) includes a central unit (12), a cooling device (14) for cooling a fluid, at least one applicator (16) for performing a non-invasive localized treatment of the fats by cold application, including a cavity, a suction conduit (18) opening up into the cavity (34) and arranged so as to suck up a bead in the cavity (34), and a transport device (20) for conducting the fluid from the central unit inside the applicator. The wall of the cavity (34) is suitable for being indirectly cooled by the cooling device and the cooling device (14) is arranged at a distance from the applicator.

### FIELD OF THE INVENTION

The present invention relates to the field of medical devices for reducing localized fatty masses by means of cold application, and in particular to a so-called “cryolipolysis” device (also known by the term “Cryolipolysis®”) which permits apoptosis of the localized adipocytes of a patient.

### BACKGROUND OF THE INVENTION

Document US 2003/0220674 in particular discloses a medical device for reducing localized fatty masses by means of cold application.

This type of medical device is designed to reduce the fatty mass by using the so-called Cryolipolysis® method which is well known to persons skilled in the art. The main steps of a cryolipolysis treatment are as follows: the area of the skin to be treated is covered with a cloth previously impregnated with a cryoprotective gel, then the applicator of the machine is placed on this cloth. The machine progressively sucks up the wad of fat into a cavity in the applicator and cools it.

The duration of the session with this machine varies from a few tens of minutes to more than an hour depending on the area to be treated. At the end of the session a wad hardened by the cold is obtained. The practitioner then carries out energetic massage of the wad in order to break up the adipocytes and improve the lymphatic drainage, and in order for the skin to adopt its normal appearance once more.

There are also known in the prior art devices for reducing localized fatty masses by means of cold application comprising a central unit and one or a plurality of standard applicators for the treatment of the localized fatty masses by means of cold application.

The central unit comprises in particular electronic boards, a control screen, a cold unit, a vacuum pump, a water pump, a cooling circuit, electrical beams, or also other elements which are known to persons skilled in the art, and are conventionally used in central unit of this type of a device for reducing fats by means of cold application.

The applicator(s) comprise(s) a cavity with a substantially rectangular form, which is designed to receive a wad of fat from a patient, cold dissipater elements, and a system for suction of the wad in order to draw it into the cavity.

More specifically, the applicator comprises a housing, an electronic board, Peltier-effect cells for generation of cold, at least two lateral header tanks provided for cooling of the hot plates of the Peltier-effect cells, and input and output connectors for the header tanks. An applicator of this type is represented in particular in FIG. 1.

Thus, the known cryolipolysis devices are particularly complex to produce, in that they require the central unit to be connected electronically and electrically to the applicators in order to trigger and control the cooling elements. In addition, in the devices according to the prior art, it is also necessary to provide temperature sensors in the vicinity of the skin which are connected to the central unit in order to determine and control the temperature level of the cooling elements.

In addition, the devices according to the prior art are provided with numerous elements inside the applicators. Thus, the design of a device of this type is complex to produce because of the large number of elements necessary for the manufacture of the applicators. Also, the assembly of the applicators of the devices according to the prior art requires a qualified workforce, and a large number of working hours is necessary in order to accommodate all the aforementioned elements inside the housing of the applicator of such devices.

Furthermore, when the devices according to the prior art comprise a safety system to prevent the patient's skin from being burned when the cooling elements are malfunctioning, this system generally comprises sensors to measure the temperature of the patient's skin in the treatment area. These sensors are mostly integrated in the applicator such as to be in contact with, or in the immediate vicinity of, the patient's skin, when the applicator is applied to the skin. This safety system is therefore complex, and increases the number of electronic elements necessary in order to ensure adequate safety. In addition, since the applicator must accommodate a plurality of temperature sensors as well as an electronic board for the operation of these sensors, its size cannot easily be reduced.

Also, it is particularly difficult to design devices comprising specific applicators for complex anatomical areas, such as very small wads in particular.

### SUMMARY OF THE INVENTION

The aim of the present invention is in particular to eliminate these disadvantages, and its objective is in particular to simplify the production of a device designed to carry out non-invasive treatment for reducing fats by means of cold application, whilst permitting better adaptation of an applicator to the morphology of the human body.

For this purpose, the invention relates to a system which is designed to carry out non-invasive treatment for reducing fats by means of cold application comprising:
a central unit comprising a control device;
a cooling device, the cooling device being designed to cool a fluid to a cooling temperature lower than 0° C., the control device controlling the cooling device;
at least one applicator which is designed to carry out a localized non-invasive treatment of the fats by means of cold application, the applicator comprising a cavity defined by a wall, said cavity being designed to receive a localized wad or mass of fat of a patient;
a suction duct which opens into the cavity, and is designed to suck the wad up into said cavity;
a transport device which is designed to convey the fluid from the central unit to the interior of the applicator,
   characterized in that the wall is designed to be cooled indirectly by the cooling device, and in that the cooling device is arranged at a distance from the applicator.

Thanks to these arrangements, a system according to the invention which is designed to carry out non-invasive treatment for reducing fats by means of cold application comprises far fewer elements inside the applicator (also known as the hand-held part), which simplifies greatly the production of the system as a whole, whilst reducing the production cost and time, and without detracting from the performance of the system. In fact, the applicator no longer comprises Peltier-effect cells or any other active element for generation of cold, and therefore an electrical element or electronic board is no longer required to control these Peltier-effect cells or any other active element for generation of cold. In addition, all of the electrical wiring for the control and power supply of the Peltier-effect cells is no longer necessary. Any electronic boards which are present in the system, and for example in the central unit, are also simpler to produce.

In addition, all the complex steps of assembly of the applicators are eliminated. In fact, the system according to the invention no longer requires a step of meticulous gluing of at least four Peltier-effect cells per applicator. It is no longer necessary for the Peltier-effect cells to be placed in a heat dissipater and a specific header tank which is particularly complex to produce because it must be able to receive the Peltier-effect cells. Also, with the system according to the invention, it is no longer necessary to follow the procedure for wiring the Peltier-effect cells with an electronic board which is integrated in the applicator. This therefore prevents any problems of compactness which compel a qualified worker to use special tools. In fact, the systems known in the prior art are generally provided with a set of 5 interchangeable applicators, and require the use of 20 Peltier-effect cells, 5 electronic boards, and many other elements such as water connectors, screws, thermal adhesive tape, and, of course, hours of complex assembly. Since the applicator of the system according to the invention is without any electronic component or active cooling device, it can easily be miniaturized, or it can be produced in different forms which are or are not complex, in order to permit better adaptation of this applicator to the morphology of certain areas of the human body. The applicators of the system according to the invention are thus potentially lighter. The absence of electric current passing through the applicator makes it possible in particular to extend its use to a larger number of patients, and in particular to patients wearing cardiac stimulators (pacemakers).

The fluid advantageously has a solidification temperature higher than −13° C., and preferably higher than −11° C. In addition or alternatively, the fluid has a solidification temperature lower than −9° C.

In addition, thanks to the device according to the invention, any risk of malfunctioning of a Peltier-effect cell (also known as Peltier cell), giving rise to particularly cold and potentially dangerous temperatures, is eliminated.

According to preferred embodiments of the invention, recourse can optionally also be made to one and/or the other of the following provisions, taken alone or in combination.

According to one embodiment, there is a single cooling device which is arranged at a distance from the applicator, such as to cool the fluid outside the applicator. The presence of a single cooling device makes it possible to simplify the production of the system, and to reduce the assembly times.

According to one embodiment, the transport device comprises a portion which is accommodated in the applicator, and the transport device is designed to convey the fluid into the applicator, such that the fluid passes through the applicator at an application temperature higher than the cooling temperature and lower than 2° C. The transport device guides the cooled fluid at a distance from the applicator, as far as into the applicator. The single cooling device can on its own cool the fluid to a temperature which is low enough for the fluid transported into the applicator to be suitable for carrying out a treatment of reducing the fats by means of cold application without adding additional Peltier-effect cells in the applicator.

According to one embodiment, the transport device comprises a portion which is arranged in direct contact with the applicator, and the transport device is designed to convey the fluid to the applicator, such that the fluid reaches the applicator at an application temperature higher than the cooling temperature and lower than 2° C.

According to one embodiment, the cooling device comprises one or a plurality of localized cooling elements at a distance of at least 50 cm from the applicator. In other words, the transport circuit which forms an intermediary between the cooling device and the applicator comprises for example a conduit which extends longitudinally over a length greater than 50 cm. Thus, there is no longer any need to place additional cooling elements in the vicinity of the applicator, which simplifies greatly its use, since the applicator is consequently more compact and lighter.

According to one embodiment, the cooling device is arranged inside the central unit. Thus, all of the cold is generated only inside the central unit, and not in the applicator, which reduces the risk of accident in particular, as well as the size of the applicator.

According to one embodiment, the system comprises electrical and electronic elements which are designed to control parameters of the system. These electrical and electronic elements are for example control boards which make it possible to automate the system or to regulate the temperature of the fluid.

According to one embodiment, the electrical and electronic elements are arranged at a distance from the applicator. Thus, the applicator does not contain any electrical or electronic element which acts on parameters of the system. For example, the electrical and electronic elements are arranged at a distance of at least 50 cm from the applicator.

According to one embodiment, the transport device comprises one or a plurality of header tanks.

According to an additional embodiment, the one or a plurality of header tanks is/are configured to cool the wall of the cavity of the applicator directly. In other words, the header tank(s) make(s) it possible to convey the fluid and thus transfer the cooled fluid in the applicator to the exterior of the applicator, such as to cool the wall of the cavity of the applicator.

According to an alternative embodiment, the transport device is partly integrated in the wall of the cavity. For example, conduits can be provided in the thickness of the wall of the cavity, such as to convey the fluid, and permit cooling of the surface of the wall of the cavity which is designed to be in contact with the skin of a patient.

According to one embodiment, the cooling device comprises a reservoir comprising a coolant fluid.

According to one embodiment, the coolant fluid is taken from amongst the list of: a solution composed of a mixture of water and propylene glycol, a solution composed of a mixture of alcohol and water. Fluids of this type make it possible to limit the risk of dropping to excessively cold temperatures, in particular by using a fluid with a known point of solidification or crystallization, for example of approximately −8° C. Thus, if malfunctioning of the system gives rise to excessive cooling of the fluid, the fluid solidifies (or gels), which prevents its circulation in the transport device, and thus avoids any risk of burning of the patient by the cold. The system thus makes it possible to limit the risk of dropping to excessively cold temperatures, in particular by using a fluid with a known point of solidification or crystallization.

According to one embodiment, the cooling device comprises Peltier-effect cells or a cold unit. The Peltier-effect cells or the cold unit form elements for cooling of the cooling device. These cooling elements are “active” elements.

The present invention also relates to an applicator which is designed to carry out a non-invasive treatment for reducing fats by means of cold application which is designed to be associated with a central unit, a cooling device and a transport device, such as to form a system which is designed to carry out a non-invasive treatment for reducing fats by means of cold application as previously described, said applicator comprising:
a portion of transport device comprising at least one header tank for the transport of a fluid, in particular a subzero fluid;
a hollow metal element comprising a wall forming a cavity, said cavity being designed to receive a localized wad or mass of fat of a patient;
a portion of suction duct opening into the cavity and arranged such as to suck the wad up into said cavity,
   characterized in that the transport device comprises header tanks arranged directly in thermal connection against the wall of the cavity which is designed to receive a localized wad or mass of fat of a patient, the applicator being without Peltier-effect cells.

An applicator of this type is in particular easy to implement, light, and can have forms which are or are not complex, in order to adapt to the morphology of a patient.

Finally, the present invention relates to a method for non-invasive treatment for reducing fats by means of cold application by the system as previously described, comprising the steps of:
suction of a wad of fat inside the metal cavity of the applicator;
cooling of a fluid inside the central unit;
transport of the fluid from the central unit to the applicator;
direct absorption of heat between a header tank and the wall of the cavity, such as to reduce the fats by means of cold application.

A method of this type makes it possible in particular to avoid any step of generation of cold in the applicator, and of checking by the sensor of the temperature of the patient's skin.

### BRIEF DESCRIPTION OF THE DRAWINGS

Other characteristics and advantages of the invention will become apparent from the following description of one of its embodiments, provided by way of non-limiting example, with reference to the appended drawings.

In the drawings:

FIG. 1 is a schematic representation of the system designed to carry out non-invasive treatment for reducing fats by means of cold application according to the invention, comprising a central unit, a cooling device, an applicator according to a first embodiment, and a suction duct;

FIG. 2 is a view from above of a header tank of the applicator in FIG. 1;

FIG. 3 is a view in cross-section of an applicator according to a second embodiment of the invention.

### MORE DETAILED DESCRIPTION

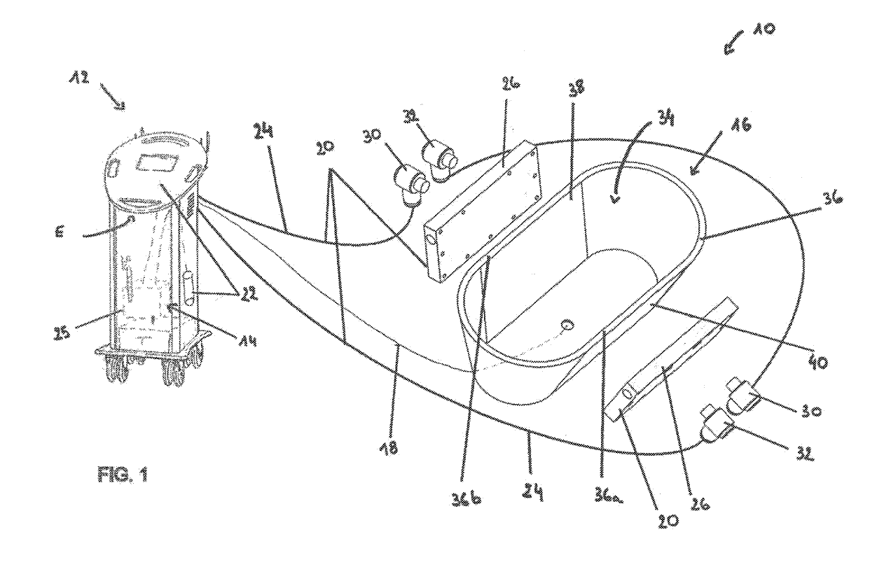

FIG. 1 represents schematically a system 10 designed to carry out non-invasive treatment for reducing fats by means of cold application, comprising a central unit 12, a cooling device 14, at least one applicator 16, a suction duct 18 and a transport device 20.

The central unit 12 comprises a control device 22, as well as all the elements habitually found in the central unit of a system which is designed to carry out non-invasive treatment for reducing fats by means of cold application known by persons skilled in the art. The central unit 12 can thus comprise a screen, which is or is not a touchscreen, for control of the system, one or a plurality of electronic boards E, a vacuum pump, a coolant fluid pump, a reservoir for coolant liquid, solenoid valves, fans, and any other elements well known by persons skilled in the art in systems for reducing fats by means of cold application.

The cooling device 14 is designed to cool a fluid to a cooling temperature lower than 0° C. The control device 22 controls the cooling device 14. More specifically, the control device 22 is designed to control parameters of the cooling device such as, for example, the temperature of the fluid, the flow rate, the cooling time, etc.

The cooling device 14 comprises for example a cold unit of the type which operates with a coolant gas, such as R134a. A cooling device 14 of this type has cold generation performance such that the fluid to be cooled can reach a particularly cold temperature of approximately −25° C. In particular, the cold unit used with a coolant gas such as R134a is sufficient to permit cooling of the fluid which is designed to pass via the transport device 20 to a satisfactory temperature at the cooling device 14 (or cold unit), in order to reach the applicator with an application temperature which is sufficient to carry out apoptosis of adipocytes in the area to be treated of the patient. For reasons of safety, the cold unit can be restrained electronically such that it can cool the fluid to temperature of −12° C.

The cooling device 14 is for example integrated in the central unit 12. In other words, the central unit 12 comprises for example a housing which in particular contains the cooling device 14, as illustrated in FIG. 1. According to a variant embodiment, the cooling device 14 can be arranged outside the central unit, in a separate housing for example.

The fluid cooled by the cooling device 14 can for example be a solution which is a mixture of water and alcohol which ensures advantageous circulation of the fluid. For example, the solution comprises 80% water for 20% alcohol. A mixture of this type has a solidification point which corresponds to a limit solidification temperature. For example, the limit solidification temperature is approximately −12° C. or −10° C. Thus, if a malfunction of the cooling device occurs and the fluid is cooled to below the limit temperature, the fluid solidifies. The solidification of the fluid prevents its free circulation in the transport device 20, which blocks the operation of the system. Thus, the patient is protected against any burning by the cold, since the system can not operate if the fluid is cooled to below a limit temperature which could give rise to burns.

The fluid cooled by the cooling device 14 can for example be a solution which is a mixture of water and polypropylene glycol. For example, the solution comprises 80% water and 20% polypropylene glycol. The limit temperature for solidification of a fluid of this type is approximately −15° C.

The minimum treatment temperature observed by the present inventors from amongst the treatments proposed at present is −13° C.

This is why, advantageously, the fluid used has a solidification temperature of −13° C. or more. Examples of such fluids are: a mixture of water and propylene glycol with a ratio of water to propylene glycol of more than 2 or 2.3; a mixture of water and ethylene glycol with a ratio of water to ethylene glycol of more than 2 or 2.2; a mixture of water to GreenWay® Neo (by Climalife® dehon) with a ratio of water to GreenWay® Neo of more than 1.8 or 2.

In addition, it seems that there is a greater consensus amongst cryolipolysis professionals not to lower the treatment temperature below −11° C. in order to prevent burning of the skin by the cold. Thus, a fluid with a solidification temperature of −11° C. or more is preferred. Examples of such fluids are: a mixture of water and propylene glycol with a ratio of water to propylene glycol of more than 2.5 or 2.7; a mixture of water and ethylene glycol with a ratio of water to ethylene glycol of more than 3.5 or 3.7; a mixture of water and GreenWay® Neo with a ratio of water to GreenWay® Neo of more than 2 or 2.3.

GreenWay® Neo is a heat exchange fluid made by the company Climalife® dehon based on 1,3-propanediol (2017).

The use of a fluid of this type makes it possible to dispense with sensors in the applicator and electronic elements for monitoring the temperature of the skin, and to put the system out of service in the event of excessively high-level running of the cooling device, and in particular of the Peltier elements. In addition, an electronic safety system is not protected against electronic malfunction.

Use of a fluid with a solidification temperature of more than −13° C. or −11° C. makes it possible to put the system out of service without having to count on electronic elements. In addition, the use of a fluid with a solidification temperature of more than −11° C. has the advantage compared with a fluid with a solidification temperature of more than −13° C. of detecting more rapidly any excessively high-level running of the cooling device.

In addition, in certain cases, such as, for example, in a cryolipolysis device which is designed for treatment of areas of the body where it is agreed that the skin is thicker and less sensitive to cold, and for which particularly low temperatures are the most efficient in order to obtain good results, it is preferable for the liquid to have a solidification temperature lower than −9° C. In this case, examples of fluids are: a mixture of water and propylene glycol with a ratio of water to propylene glycol lower than 3.5 or 3.3; a mixture of water and ethylene glycol with a ratio of water to ethylene glycol lower than 4.7 or 4.5; a mixture of water and GreenWay® Neo with a ratio of water to GreenWay® Neo lower than 3.5 or 3.

The fluid which is cooled by the cooling device 14 is conveyed to the applicator 16 by means of the transport device 20. The transport device 20 comprises for example a main duct 24. The main duct 24 is for example made of a plastic material, for example a braided plastic material, and comprises an insulating sheath which extends around its entire periphery. The main sheath 24 extends for example longitudinally over a length of more than 50 cm. The main duct 24 extends for example longitudinally over a length of 1 m to 2 m. The main duct 24 can for example have a substantially circular cross section with a diameter of between 12 mm and 24 mm The transport device 20 is thus designed to convey the fluid from the central unit 12 to the interior of the applicator. More specifically, the transport device 20 comprises the main duct (which can be in the form of a flexible tube for example) extending from a reservoir 25 which is provided for example in the central unit 12. The reservoir 25 is connected to the cooling device 14, such that the fluid which is present in the reservoir is directly cooled by the cooling device 14. A temperature sensor can be provided at the input of the transport device 20, or at the output from the cooling device, in order to measure the temperature of the fluid. For example, the cooling device 14 cools the fluid to a cooling temperature. The cooling temperature is lower than 0° C. The cooling temperature can also be lower than −5° C. The cooling temperature can also be lower than −8° C.

In this case, as illustrated in FIG. 1, and according to a first embodiment, the transport device comprises two main ducts 24 which extend from the cooling device 14, and more specifically from the central unit 12, to the applicator 16. The main ducts 24 are designed to convey the fluid cooled by the cooling device 14 to the applicator 16. As represented in FIG. 1, the transport device 20 also comprises two header tanks 26. The main ducts 24 are connected to the applicator by means of two header tanks 26 which permit thermal transmission from the fluid to the applicator 16.

In variant embodiments not represented, the transport device can comprise one or more than two main ducts. In addition, the transport device can comprise one or a plurality of boxes, for example 3 or 4 header tanks.

The header tanks 26 are connected directly to the applicator 16.

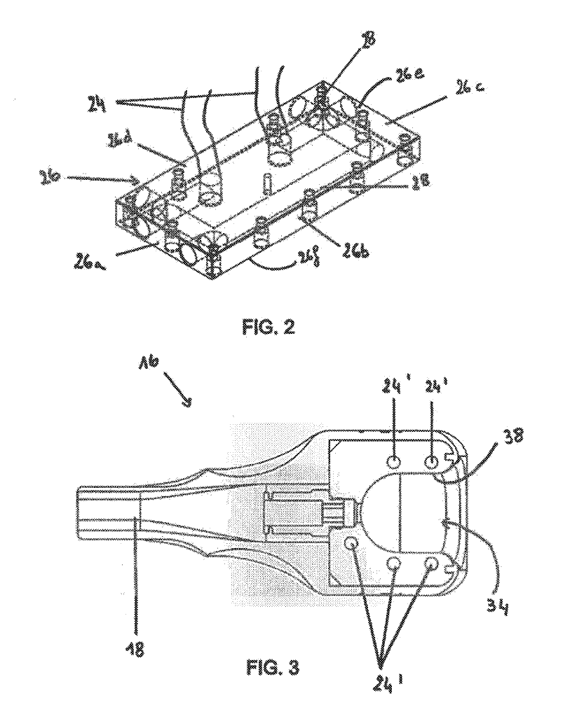

FIG. 2 illustrates according to a view in perspective a header tank 26 in FIG. 1. The header tank 26 comprises an inner cooling duct 28 for circulation of the cooling fluid by means of a fluid circulation circuit.

The header tank 26 forms a block (for example with a parallelepiped form) provided with a plurality of faces delimited by ridges, and one of the largest faces of which comprises an orifice for an input connector 30 (shown in FIG. 1) for the cooling fluid, and an orifice for output of the cooling fluid via an output connector 32, each of the orifices being designed to supply the fluid circulation circuit of the header tank.

The header tank 26 is for example made of aluminum. The header tank 26 comprises for example eight faces and has a thickness of approximately 12 mm. As illustrated in FIG. 2, the header tank 26 comprises four lateral faces 26a, 26b, 26c, 26d, and two main faces 26e, 26f, all delimited by straight ridges. The header tank 26 comprises lateral sections which are provided with production holes necessary for creation of the fluid circulation circuit which is arranged inside the header tank 26. The header tank 26 comprises receptacles for screws or other securing units for assembly of the header tank on the applicator 16.

As can be seen in FIG. 1, the applicator 16 comprises in particular a cavity 34 defined by a wall 36. The cavity 34 is designed to receive a localized wad or mass of fat of a patient. The wall 36 comprises an inner surface 38 and an outer surface 40, opposite the inner surface 38. The inner surface 38 is oriented towards the cavity 34, and in this case the inner surface 38 defines the contours of the cavity 36. The wall 36 of the applicator 16 comprises two longitudinal portions 36a, 36b which are connected at their ends by rounded portions 36c, 36d, such as to have a substantially frusto-conical form. The main faces 26e, 26f of the header tank 26 are disposed substantially parallel to the longitudinal portions 36a, 36b. FIG. 1 represents a single applicator. However, in variant embodiments, a plurality of applicators can be provided.

The wall 36 of the applicator is for example made of metal. Metal permits good thermal conductivity. Thus, the fluid is transported by the transport device 20 firstly into the main duct(s) 24, then into the header tank(es) 26, which is/are arranged directly in thermal connection against the wall of the cavity. The fluid reaches the inside of the applicator or the direct vicinity of the applicator at an application temperature. In this case, the application temperature is slightly higher than the cooling temperature. This is because of the energy losses of the liquid in the transport device 20. For example, if the cooling temperature is lower than 0° C., the cooling temperature can be lower than 2° C. Optionally, if the cooling temperature is lower than −5° C., the cooling temperature can be lower than 0° C. or −1° C. The energy loss is between 1 and 3° C. for a main duct 24 of between 1 and 2 m. In addition, the fluid selected (mixture of water and alcohol in particular) associated with a sufficiently powerful cooling device makes it possible to cool the fluid at a distance from the applicator which is sufficient to avoid having to cool the fluid again inside the applicator. In addition, fluids such as those previously mentioned have relatively low energy losses. Thus, the temperature of the fluid cooled at the central unit is sufficient to carry out apoptosis of the adipocytes of the area of the patient which is situated in the cavity.

Into the cavity 34 there opens the suction duct 18 which is designed to suck up a localized wad or mass of fat of the patient, such as to retain the wad or mass of fat inside the cavity, so that the wall of the cavity, cooled by means of the fluid circulating in the transport device 20, itself cools the wad or mass of fat of the patient which is retained in the cavity. The suction duct 18 consists for example of a flexible conduit which is connected to a blower. The blower can be arranged inside the central unit 12 for example. According to a variant embodiment, the suction of the wad into the cavity 34 can be carried out by a system of suckers.

According to a second embodiment of the transport device 20 illustrated in particular partly in FIG. 3, a portion of the transport device is integrated in the wall of the cavity, and substantially surrounds the cavity. In this case, the transport device 20 comprises one or a plurality of main ducts 24 which are extended by secondary ducts 24′. The secondary ducts 24′, as illustrated in FIG. 3, are accommodated in the wall 36 of the applicator 16. In other words, the secondary ducts 24′ are integrated in the wall 36 of the applicator 16. The fluid obtained from the main duct(s) also circulates in the secondary ducts 24′, such as to cool the inner surface 38. As represented in FIG. 3, a plurality of secondary ducts are provided. However, according to variant embodiments, a single secondary duct can be provided, which is wound around the cavity 34. The applicator 16 as represented in FIG. 3 has a form slightly different from that of the applicator 16 represented in FIG. 1. In this case, the form of the applicator can depend on the form of the cavity 34 of the applicator required. The form of the cavity 34 can depend on the area to be treated of the patient. Thus, the cavity 34 can have any form, which is or is not complex, and adapts to the morphology of the area to be treated of the patient.

The applicator 16 connected to the transport device 20 according to the first embodiment represented in FIG. 1, or according to the second embodiment represented in FIG. 3, comprises a portion of transport device comprising at least one header tank for the transport of a fluid, in particular a subzero fluid. The applicator 16 also comprises a portion of suction duct which opens into the cavity. However, the applicator does not comprise any “active” cooling device. The wall of the applicator is cooled by absorption of direct heat between the wall, which in this case is made of metal, and the fluid circulating in the transport device 20. The applicator is without any generator of cold, Peltier-effect cells, or any electrical or electronic component which is associated directly or indirectly with the generation of a low temperature. In other words, no component for generation of cold is provided inside the applicator. The applicator 16 is thus means or an element or device for cooling. The cooling device which permits cooling of the wall 36 of the applicator indirectly is accommodated outside the applicator and at a distance from it.

The system 10 can also comprise elements for insulation of the “cold” equipment, such as to prevent any risk of burning by the cold, and to prevent substantial energy losses of the fluid on the path between the central unit 12 and the applicator 16. For example, the transport device 20 comprises an insulator along the entire length of the main duct 24.

The system 10 which is designed to carry out a non-invasive treatment for reducing fats by means of cold application can be used in the manner described hereinafter.

In one step, a mass of fat is sucked up inside the cavity 34 of the applicator. As previously stated, the cavity can be formed by a metal wall 36 for better thermal conductivity.

In a subsequent step or before the suction, cooling takes place of a fluid provided inside a reservoir. For example, the reservoir can be arranged inside the central unit.

In one step, the fluid is transported from the reservoir 25 to the applicator 16 by means of the transport device 20.

In one step, the heat of the wall which forms the cavity 34 is absorbed, for example by a header tank, such as to permit cooling of the wad accommodated in the cavity, and thus reduce the fat by means of cold application.

No step of generation of cold inside the applicator is necessary, for example by Peltier-effect cells.

1.-14. (canceled)

15. A non-invasive fat reducing system (10) which is designed to carry out non-invasive treatment for reducing fats by means of cold application comprising:
a central unit (12) comprising a controller (22);
a cooler (14), the cooler (14) being designed to cool a fluid to a cooling temperature lower than 0° C., the controller (22) controlling the cooler (14);
at least one applicator (16) which is designed to carry out a localized non-invasive treatment of the fats by application of cold, the applicator (16) comprising a cavity (34) defined by a wall (36), said cavity (34) being designed to receive a localized wad or mass of fat of a patient;
a suction duct (18) which opens into the cavity (34), and is designed to suck the wad up into said cavity (34);
a transporter (20) which is designed to convey the fluid from the central unit (12) to the interior of the applicator (16),
wherein the wall (36) is designed to be cooled indirectly by the cooler (14), and in that the cooler (14) is arranged at a distance from the applicator (16).

16. The non-invasive fat reducing system (10) as claimed in claim 15, wherein the fluid has a solidification temperature higher than −13° C.

17. The non-invasive fat reducing system (10) as claimed in claim 16, wherein the fluid has a solidification temperature higher than −11° C.

18. The non-invasive fat reducing system (10) as claimed in claim 15, wherein the fluid has a solidification temperature lower than −9° C.

19. The non-invasive fat reducing system (10) as claimed in claim 15, wherein there is a single cooler (14) which is arranged at a distance from the applicator (16), such as to cool the fluid outside the applicator (16), and wherein the transporter comprises a portion which is accommodated in the applicator (16), the transporter (20) being designed to convey the fluid into the applicator (16), such that the fluid passes through the applicator (16) at an application temperature higher than the cooling temperature and lower than 2° C.

20. The non-invasive fat reducing system (10) as claimed in claim 15, wherein the cooler (14) comprises one or a plurality of localized cooling elements at a distance of at least 50 cm from the applicator (16).

21. The non-invasive fat reducing system (10) as claimed in claim 15, wherein the cooler (14) is arranged inside the central unit (12).

22. The non-invasive fat reducing system (10) as claimed in claim 15, comprising electrical and electronic elements (E) which are designed to control parameters of the system (10), and wherein the electrical and electronic elements (E) are arranged at a distance from the applicator (16).

23. The non-invasive fat reducing system (10) as claimed in claim 15, wherein the transporter (20) comprises one or a plurality of header tanks (26), and wherein the one or a plurality of header tanks (26) is/are configured to cool the wall (36) of the cavity (34) of the applicator (16) directly.

24. The non-invasive fat reducing system (10) as claimed in claim 15, wherein the transporter (20) is partly integrated in the wall (36) of the cavity (34).

25. The non-invasive fat reducing system (10) as claimed in claim 15, wherein the cooler (14) comprises a reservoir (25) comprising a coolant fluid, and wherein the coolant fluid is chosen from the list consisting of: a solution composed of a mixture of water and propylene glycol, a solution composed of a mixture of alcohol and water.

26. The non-invasive fat reducing system (10) as claimed in claim 15, wherein the cooler (14) comprises Peltier-effect cells or a cold unit.

27. An applicator (16) which is designed to carry out a non-invasive treatment for reducing fats by means of cold application and is adapted especially to be associated with a central unit (12), a cooler (14) and a transporter (20), such as to form a system (10) which is designed to carry out a non-invasive treatment for reducing fats by means of cold application as claimed in claim 15, said applicator (16) comprising:
a portion of transporter (20) comprising at least one header tank (26) for the transport of a fluid, in particular a subzero fluid;
a hollow metal element comprising a wall (36) forming a cavity (34), said cavity (34) being designed to receive a localized wad or mass of fat of a patient;
a portion of suction duct (18) opening into the cavity (34) and arranged such as to suck the wad up into said cavity (34),
wherein the transporter (20) comprises header tanks (26) arranged directly in thermal connection against the wall (36) of the cavity (34) which is designed to receive a localized wad or mass of fat of a patient, the applicator (16) being without Peltier-effect cells.

28. A method for non-invasive treatment for reducing fats by means of cold application by a system (10) as claimed in claim 15, comprising the steps of:
suction of a wad of fat inside the metal cavity (34) of the applicator (16);
cooling of a fluid inside the central unit (12);
transport of the fluid from the central unit (12) to the applicator (16);
direct absorption of heat between a header tank (26) and the wall (36) of the cavity (34), such as to reduce the fats by means of cold application.
